(19) United States
(12) Patent Application Publication
(10) Pub. No.: US 2026/0199562 A1
(43) Pub. Date: Jul. 16, 2026

(54) TEMPERATURE-SENSITIVE DEGRADABLE GEL FOR TREATING INTRAUTERINE ADHESION AND PREPARATION METHOD THEREOF

(71) Applicant: XIANGYA HOSPITAL CENTRAL SOUTH UNIVERSITY, Changsha (CN)

(72) Inventors: Nianchun SHAN, Changsha (CN); Wei ZHOU, Changsha (CN); Wei HUANG, Changsha (CN); Xinyi LIU, Changsha (CN); Li PAN, Changsha (CN)

(73) Assignee: XIANGYA HOSPITAL CENTRAL SOUTH UNIVERSITY, Changsha (CN)

(21) Appl. No.: 19/435,896

(22) Filed: Dec. 30, 2025

Related U.S. Application Data
(63) Continuation of application No. PCT/CN2025/086071, filed on Apr. 21, 2025.

(30) Foreign Application Priority Data
Jan. 15, 2025 (CN) 2025100608119

(51) Int. Cl. A61L 31/04; A61L 31/00; A61L 31/14; C08L 5/06; C08L 5/08; C08L 5/14; C08L 89/00

(52) U.S. Cl. CPC A61L 31/041; A61L 31/005; A61L 31/148; C08L 5/06; C08L 5/08; C08L 5/14; C08L 89/00; A61L 2300/418

(57) ABSTRACT

This disclosure provides a temperature-sensitive degradable gel for treating intrauterine adhesion and a preparation method thereof, belonging to the technical field of gels. The preparation method includes the following steps: preparation of an amygdalin-loaded polymer; preparation of hydroxyproline-modified chitosan; and mixed preparation of the temperature-sensitive degradable gel. According to this disclosure, soybean protein isolates and carboxymethyl konjac glucomannan are first prepared into solutions, respectively, amygdalin is added to the soybean protein isolate solution, and then a resulting mixture is mixed with the carboxymethyl konjac glucomannan solution for electrostatic self-assembly polymerization to form a carrier with a three-dimensional macromolecular structure, the amygdalin is loaded, and thus an amygdalin-loaded polymer is obtained. After the amygdalin-loaded polymer is added to the gel, the deformation resistance of the gel can be enhanced, thereby achieving the effect of improving the support force of the gel and reducing a recurrence rate of the intrauterine adhesion.

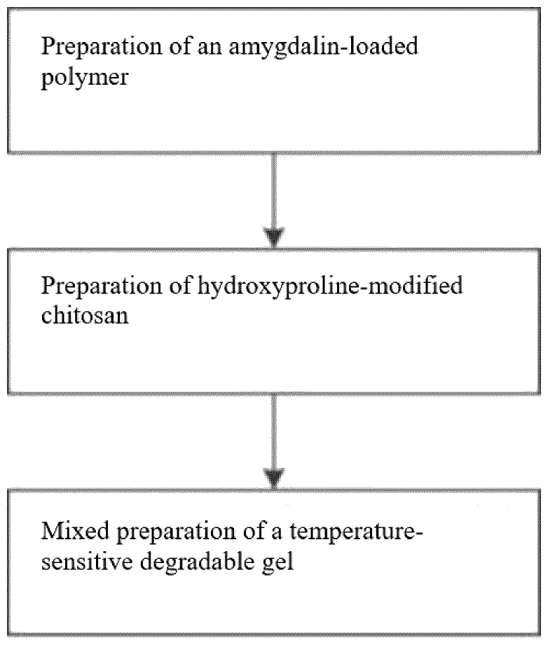

### CROSS-REFERENCE TO RELATED APPLICATIONS

The application claims priority to Chinese patent application No. 2025100608119, filed on Jan. 15, 2025, the entire contents of which are incorporated herein by reference.

### TECHNICAL FIELD

This disclosure relates to the technical field of gels, and in particular, to a temperature-sensitive degradable gel for treating intrauterine adhesion and a preparation method thereof.

### BACKGROUND

Intrauterine adhesion is a common gynecological disorder, often caused by local surgical procedures for the uterine cavity (such as induced abortion, curettage, etc.), inflammatory infections, and other factors which lead to damage of the uterine cavity mucosa and subsequently result in uterine cavity stenosis or atresia. This pathological state severely impacts women's reproductive health, potentially causing irregular menstruation such as reduced menstrual flow or even amenorrhea, as well as fertility disorders including infertility and recurrent abortions. Statistics indicate that among women who have undergone multiple intrauterine operations, the incidence of intrauterine adhesion shows a rising trend year by year, making it one of the most important issues to be urgently addressed in gynecological clinical practice.

Conventional treatment for intrauterine adhesion mainly relies on surgical separation of adhesion tissues, such as hysteroscopic adhesiolysis. Although this method can directly remove the adhesion sites, the risk of postoperative re-adhesion in the uterine cavity is extremely high. This is due to the lack of effective physical barriers and tissue support during the healing process of the surgical wounds. The uterine cavity mucosa is prone to re-adhesion before complete repair, leading to adhesion recurrence. As a result, multiple repeated surgeries are often required, which brings great physical pain and psychological burden to patients, while also increasing medical costs and consuming more medical resources. Drug treatment primarily focuses on preventing infection and promoting endometrial repair, e.g., using antibiotics to prevent infection and applying estrogen to promote endometrial growth. However, simple drug treatment is highly limited in their ability to improve the physical structure of the uterine cavity and provide support. In the case that the morphology of the uterine cavity is not effectively maintained, it is difficult for drugs to establish an effective local concentration gradient and functional environment. Consequently, their effect on the endometrial repair is greatly reduced, failing to fundamentally address the critical issue of intrauterine adhesion recurrence.

In contrast, as a novel material for local drug delivery and tissue repair, the temperature-sensitive gel can act more precisely on the adhesion site compared with the drug treatment. Under the effect of body temperature, the temperature-sensitive gel undergoes gelation, allowing the drugs or treatment components to remain concentrated on the adhesion site for a long time, thereby enhancing the pertinence of treatment. Moreover, compared with surgical treatment, the temperature-sensitive gel provides a non-invasive physical barrier approach, which can form a physical barrier layer in the uterine cavity, effectively prevent the separated uterine cavity tissues from contacting again, and reduce the risk of adhesion recurrence. However, existing gels are prone to the issue of insufficient support force. In the physiological environment of the uterine cavity, due to the influence of human activities, tissue peristalsis, gravity, and other factors, gels with weak support force cannot stably maintain the open morphology of the uterine cavity. Such gels are prone to deformation and collapse when subjected to even a small external force, failing to provide sufficient space and stable physical support for the repair of the uterine cavity mucosa. As a result, such gels cannot effectively reduce the recurrence rate of adhesion, which limits their wide application in the treatment of intrauterine adhesion.

Therefore, it is necessary to propose a temperature-sensitive degradable gel with large support force for treating intrauterine adhesion and a preparation method thereof, so as to improve the treatment effect and reduce the recurrence rate.

### SUMMARY

In view of the defects in the prior art, the objective of this disclosure is to provide a temperature-sensitive degradable gel for treating intrauterine adhesion and a preparation method thereof.

A preparation method of a temperature-sensitive degradable gel for treating intrauterine adhesion, including the following steps:

### S1: Preparation of an Amygdalin-Loaded Polymer

preparing a soybean protein isolate solution and a carboxymethyl konjac glucomannan solution from soybean protein isolates and carboxymethyl konjac glucomannan, respectively, adding amygdalin to the soybean protein isolate solution, and then mixing with the carboxymethyl konjac glucomannan solution for polymerization to obtain the amygdalin-loaded polymer;

### S2: Preparation of Hydroxyproline-Modified Chitosan

dissolving and activating hydroxyproline, then adding to an acetic acid solution of chitosan, followed by adding triethylamine and performing stirring reaction to obtain the hydroxyproline-modified chitosan; and

### S3: Mixed Preparation of the Temperature-Sensitive Degradable Gel

dissolving polygalacturonic acid, sodium β-glycerophosphate, and the hydroxyproline-modified chitosan, respectively, stirring and mixing same in an ice-water bath, then adding autologous platelet-rich plasma and the amygdalin-loaded polymer, and performing homogeneous mixing to obtain the temperature-sensitive degradable gel.

Further, S1 specifically includes the following steps:
S1.1: adding the soybean protein isolates and the carboxymethyl konjac glucomannan to distilled water respectively at a material-liquid ratio of 1 g:(90-100) mL, heating and stirring at 35-45° C. for dissolving while maintaining temperature, so as to obtain the soybean protein isolate solution and the carboxymethyl konjac glucomannan solution;
S1.2: adding hydrochloric acid to the soybean protein isolate solution to adjust pH to 3-4, then adding the amygdalin at a solid-liquid ratio of 1 g:(320-330) mL, and performing ultrasonic processing at 160-180 W for 20-30 min to obtain an amygdalin mixture; and
S1.3: adding the carboxymethyl konjac glucomannan solution to the amygdalin mixture, stirring at a rate of 700-800 r/min for 1-2 h, and performing polymerization to obtain the amygdalin-loaded polymer.

Further, S2 specifically includes the following steps:
S2.1: adding the chitosan to a 1% acetic acid solution at a solid-liquid ratio of 1 g:(80-90) mL, and fully stirring for dissolving to obtain a chitosan solution;
S2.2: adding the hydroxyproline to deionized water at a solid-liquid ratio of 1 g:(10-20) mL, and fully stirring for dissolving to obtain a hydroxyproline solution;
S2.3: adding 1-ethyl-3-(3-dimethylaminopropyl) carbodiimide hydrochloride and N-hydroxysuccinimide to the hydroxyproline solution, performing stirring reaction in an ice-water bath at 1-3° C. for 1-2 h to obtain an activated hydroxyproline solution; and
S2.4: adding the activated hydroxyproline solution to the chitosan solution while stirring, then adding the triethylamine, adjusting pH to 7-8, continuing to perform stirring reaction for 16-20 h, and then performing dialysis with the deionized water and freeze-drying to obtain the hydroxyproline-modified chitosan.

Further, S3 specifically includes the following steps:
S3.1: adding the hydroxyproline-modified chitosan prepared in step S2.4 to a 1% acetic acid solution at a solid-liquid ratio of 1 g:(50-60) mL, and fully stirring for dissolving to obtain a hydroxyproline-modified chitosan solution;
S3.2: adding the polygalacturonic acid and the sodium β-glycerophosphate to the deionized water, respectively, and stirring until completely dissolved to prepare 1 wt % of a polygalacturonic acid solution and 1 wt % of a sodium β-glycerophosphate solution;
S3.3: stirring equal volumes of the hydroxyproline-modified chitosan solution, the polygalacturonic acid solution, and the sodium β-glycerophosphate solution in an ice-water bath at 2-4° C. for 20-30 min to obtain a mixed sol solution; and
S3.4: adding the autologous platelet-rich plasma and the amygdalin-loaded polymer prepared in step S1.3 to the mixed sol solution, fully stirring and mixing, and then performing homogenization processing in a homogenizer for 20-30 min to obtain the temperature-sensitive degradable gel.

Further, a volume ratio of the amygdalin mixture to the carboxymethyl konjac glucomannan solution is (2-3): 1.

Further, mass ratios of the 1-ethyl-3-(3-dimethylaminopropyl) carbodiimide hydrochloride and the N-hydroxysuccinimide to the hydroxyproline are 1:(2-3) and 1: (3-5), respectively, and a volume ratio of the activated hydroxyproline solution to the chitosan solution is 1:(8-9).

Further, a volume ratio of the autologous platelet-rich plasma to the mixed sol solution is 1:(8-10), and a mass ratio of the amygdalin-loaded polymer to the autologous platelet-rich plasma is (3-5): 1.

Further, preparation of the autologous platelet-rich plasma includes the following steps: first, collecting peripheral blood of a patient for anticoagulation treatment, and then performing centrifugation to separate the peripheral blood into an upper layer, a middle layer, and a lower layer; subsequently, aspirating the upper layer and the middle layer for secondary centrifugation; discarding supernatant, then adding thrombin and 8-10 wt % of a calcium chloride solution to a remaining portion to obtain the autologous platelet-rich plasma, where a volume percentage content of the calcium chloride solution in the autologous platelet-rich plasma is 8-12%, and a content of the thrombin therein is 15-25 U/mL.

Further, a temperature-sensitive degradable gel for treating intrauterine adhesion, where the temperature-sensitive degradable gel is prepared by the preparation method of a temperature-sensitive degradable gel for treating intrauterine adhesion described in any one of the above items.

Compared with the prior art, this disclosure has at least the following beneficial effects:

1. According to this disclosure, soybean protein isolates and carboxymethyl konjac glucomannan are first prepared into solutions, respectively, amygdalin is added to the soybean protein isolate solution, and then a resulting mixture is mixed with the carboxymethyl konjac glucomannan solution for electrostatic self-assembly polymerization to form a carrier with a three-dimensional macromolecular structure, the amygdalin is loaded, and thus an amygdalin-loaded polymer is obtained. After being added to the gel, on the one hand, the amygdalin-loaded polymer can interweave with polymer molecular chains in the gel, acting as a “reinforcement phase” embedded into a network structure of the gel, making the network structure of the gel more compact and robust; and on the other hand, its rigid structure can play a role of “blocking” and “fixing” between the molecular chains, making it more complex for the gel molecular chains to withstand external force, such that the deformation resistance of the gel is enhanced, and the effect of improving the support force of the gel is achieved.

2. According to this disclosure, through operations that hydroxyproline and chitosan are first dissolved, respectively, hydroxyproline is then activated using 1-ethyl-3-(3-dimethylaminopropyl) carbodiimide hydrochloride and N-hydroxysuccinimide, and then the activated hydroxyproline solution is mixed with the chitosan solution for reaction, chitosan is modified; and after hydroxyproline-modified chitosan is added to the gel, its rich hydrogen bond formation sites can quickly form hydrogen bonds with other components in a gel system. In a process of temperature rise, these additional hydrogen bonds can accelerate crosslinking between the molecular chains, enabling the gel to form a three-dimensional network structure in a shorter time, thereby improving the rapid gel-forming ability of the gel.

3. When the temperature-sensitive degradable gel of this disclosure is used to treat intrauterine adhesion, the autologous platelet-rich plasma and amygdalin act as its main drug components. The autologous platelet-rich plasma can stimulate the proliferation and migration of fibroblasts in the uterine cavity, promote the synthesis of an extracellular matrix, and facilitate the filling and repair of damaged tissues. When combined, the anti-inflammatory action of amygdalin and the tissue repair effect of the autologous platelet-rich plasma can complement each other. During an initial inflammatory phase, amygdalin mitigates the inflammatory response. Once inflammation-induced oxidative stress and cell damages are alleviated, growth factors within the autologous platelet-rich plasma can more effectively act upon uterine cavity tissue cells, which promotes the proliferation and differentiation of the cells, accelerate the repair of the damaged tissues, and prevent further development of the adhesion. Moreover, the growth factors in the autologous platelet-rich plasma promote the formation of new blood vessels at intrauterine adhesion sites. Amygdalin can improve the nutrient intake and metabolic status of the cells, enabling the new blood vessels to better perform their nutrient transport function, providing sufficient nutrients for the tissues under repair, and further promoting the regeneration of uterine cavity tissues and the separation of the adhesion. Therefore, the autologous platelet-rich plasma and amygdalin can synergistically treat the intrauterine adhesion, thus further improving the effect of treating the intrauterine adhesion.

### BRIEF DESCRIPTION OF THE DRAWINGS

The accompanying drawing incorporated herein and forming a part of the specification illustrates examples of this disclosure, and together with the specification, further serves to explain the principles of this disclosure and enables those skilled in the relevant art to implement and use this disclosure.

FIGURE is a flowchart for a preparation method of a temperature-sensitive degradable gel for treating intrauterine adhesion adopted by examples of this disclosure.

### DETAILED DESCRIPTION OF THE EMBODIMENTS

The following is a detailed description of a temperature-sensitive degradable gel for treating intrauterine adhesion and a preparation method thereof provided by this disclosure in combination with the accompanying drawing and specific examples.

### Example 1

As shown in the FIGURE, a preparation method of a temperature-sensitive degradable gel for treating intrauterine adhesion, including the following steps:

### S1: Preparation of an Amygdalin-Loaded Polymer

S1.1: adding soybean protein isolates and carboxymethyl konjac glucomannan to distilled water respectively at a material-liquid ratio of 1 g:90 mL, heating and stirring at 35° C. for dissolving while maintaining temperature, so as to obtain a soybean protein isolate solution and a carboxymethyl konjac glucomannan solution;
S1.2: adding hydrochloric acid to the soybean protein isolate solution to adjust pH to 3, then adding amygdalin at a solid-liquid ratio of 1 g:320 mL, and performing ultrasonic processing at 160 W for 20 min to obtain an amygdalin mixture; and
S1.3: adding the carboxymethyl konjac glucomannan solution to the amygdalin mixture, stirring at a rate of 700 r/min for 1 h, and performing polymerization to obtain an amygdalin-loaded polymer, where a volume ratio of the amygdalin mixture to the carboxymethyl konjac glucomannan solution was 2:1;

### S2: Preparation of Hydroxyproline-Modified Chitosan

S2.1: adding chitosan to a 1% acetic acid solution at a solid-liquid ratio of 1 g:80 mL, and fully stirring for dissolving to obtain a chitosan solution;
S2.2: adding hydroxyproline to deionized water at a solid-liquid ratio of 1 g:10 mL, and fully stirring for dissolving to obtain a hydroxyproline solution;
S2.3: adding 1-ethyl-3-(3-dimethylaminopropyl) carbodiimide hydrochloride and N-hydroxysuccinimide to the hydroxyproline solution, performing stirring reaction in an ice-water bath at 1° C. for 1 h to obtain an activated hydroxyproline solution, where mass ratios of the 1-ethyl-3-(3-dimethylaminopropyl) carbodiimide hydrochloride and the N-hydroxysuccinimide to the hydroxyproline were 1:2 and 1:3, respectively; and
S2.4: adding the activated hydroxyproline solution to the chitosan solution while stirring, then adding triethylamine, adjusting pH to 7, continuing to perform stirring reaction for 16 h, and then performing dialysis with the deionized water and freeze-drying to obtain the hydroxyproline-modified chitosan, where a volume ratio of the activated hydroxyproline solution to the chitosan solution was 1:8;

### S3: Mixed Preparation of the Temperature-Sensitive Degradable Gel

S3.1: adding the hydroxyproline-modified chitosan prepared in step S2.4 to a 1% acetic acid solution at a solid-liquid ratio of 1 g:50 mL, and fully stirring for dissolving to obtain a hydroxyproline-modified chitosan solution;
S3.2: adding the polygalacturonic acid and the sodium β-glycerophosphate to the deionized water, respectively, and stirring until completely dissolved to prepare 1 wt % of a polygalacturonic acid solution and 1 wt % of a sodium β-glycerophosphate solution;
S3.3: stirring equal volumes of the hydroxyproline-modified chitosan solution, the polygalacturonic acid solution, and the sodium β-glycerophosphate solution in an ice-water bath at 2° C. for 20 min to obtain a mixed sol solution; and
S3.4: adding the autologous platelet-rich plasma and the amygdalin-loaded polymer prepared in step S1.3 to the mixed sol solution, fully stirring and mixing, and then performing homogenization processing in a homogenizer for 20 min to obtain the temperature-sensitive degradable gel, where a volume ratio of the autologous platelet-rich plasma to the mixed sol solution was 1:8, and a mass ratio of the amygdalin-loaded polymer to the autologous platelet-rich plasma was 3:1; and preparation of the autologous platelet-rich plasma included the following steps: first, collecting peripheral blood of a patient for anticoagulation treatment, and then performing centrifugation to separate the supernatant, then adding thrombin and 8 wt % of a calcium chloride solution to a percentage content of the calcium chloride solution in the autologous platelet-rich plasma was 8%, and a content of the thrombin therein was 15 U/mL.

### Example 2

As shown in the FIGURE, a preparation method of a temperature-sensitive degradable gel for treating intrauterine adhesion, including the following steps:

### S1: Preparation of an Amygdalin-Loaded Polymer

S1.1: adding soybean protein isolates and carboxymethyl konjac glucomannan to distilled water respectively at a material-liquid ratio of 1 g:95 mL, heating and stirring at 40° C. for dissolving while maintaining temperature, so as to obtain a soybean protein isolate solution and a carboxymethyl konjac glucomannan solution;
S1.2: adding hydrochloric acid to the soybean protein isolate solution to adjust pH to 3.5, then adding amygdalin at a solid-liquid ratio of 1 g:325 mL, and performing ultrasonic processing at 170 W for 25 min to obtain an amygdalin mixture; and
S1.3: adding the carboxymethyl konjac glucomannan solution to the amygdalin mixture, stirring at a rate of 750 r/min for 1.5 h, and performing polymerization to obtain an amygdalin-loaded polymer, where a volume ratio of the amygdalin mixture to the carboxymethyl konjac glucomannan solution was 2.5:1;

### S2: Preparation of Hydroxyproline-Modified Chitosan

S2.1: adding chitosan to a 1% acetic acid solution at a solid-liquid ratio of 1 g:85 mL, and fully stirring for dissolving to obtain a chitosan solution;
S2.2: adding hydroxyproline to deionized water at a solid-liquid ratio of 1 g:15 mL, and fully stirring for dissolving to obtain a hydroxyproline solution;
S2.3: adding 1-ethyl-3-(3-dimethylaminopropyl) carbodiimide hydrochloride and N-hydroxysuccinimide to the hydroxyproline solution, performing stirring reaction in an ice-water bath at 2° C. for 1.5 h to obtain an activated hydroxyproline solution, where mass ratios of the 1-ethyl-3-(3-dimethylaminopropyl) carbodiimide hydrochloride and the N-hydroxysuccinimide to the hydroxyproline were 1:2.5 and 1:4, respectively; and
S2.4: adding the activated hydroxyproline solution to the chitosan solution while stirring, then adding the triethylamine, adjusting pH to 7.5, continuing to perform stirring reaction for 18 h, and then performing dialysis with the deionized water and freeze-drying to obtain the hydroxyproline-modified chitosan, where a volume ratio of the activated hydroxyproline solution to the chitosan solution was 1:8.5;

### S3: Mixed Preparation of the Temperature-Sensitive Degradable Gel

S3.1: adding the hydroxyproline-modified chitosan prepared in step S2.4 to a 1% acetic acid solution at a solid-liquid ratio of 1 g:55 mL, and fully stirring for dissolving to obtain a hydroxyproline-modified chitosan solution;
S3.2: adding the polygalacturonic acid and the sodium β-glycerophosphate to the deionized water, respectively, and stirring until completely dissolved to prepare 1 wt % of a polygalacturonic acid solution and 1 wt % of a sodium β-glycerophosphate solution;
S3.3: stirring equal volumes of the hydroxyproline-modified chitosan solution, the polygalacturonic acid solution, and the sodium β-glycerophosphate solution in an ice-water bath at 3° C. for 25 min to obtain a mixed sol solution; and
S3.4: adding the autologous platelet-rich plasma and the amygdalin-loaded polymer prepared in step S1.3 to the mixed sol solution, fully stirring and mixing, and then performing homogenization processing in a homogenizer for 25 min to obtain the temperature-sensitive degradable gel, where a volume ratio of the autologous platelet-rich plasma to the mixed sol solution was 1:9, and a mass ratio of the amygdalin-loaded polymer to the autologous platelet-rich plasma was 4:1; and preparation of the autologous platelet-rich plasma included the following steps: first, collecting peripheral blood of a patient for anticoagulation treatment, and then performing centrifugation to separate the supernatant, then adding thrombin and 9 wt % of a calcium chloride solution to a percentage content of the calcium chloride solution in the autologous platelet-rich plasma was 10%, and a content of the thrombin therein was 20 U/mL.

### Example 3

As shown in the FIGURE, a preparation method of a temperature-sensitive degradable gel for treating intrauterine adhesion, including the following steps:

### S1: Preparation of an Amygdalin-Loaded Polymer

S1.1: adding soybean protein isolates and carboxymethyl konjac glucomannan to distilled water respectively at a material-liquid ratio of 1 g:100 mL, heating and stirring at 45° C. for dissolving while maintaining temperature, so as to obtain a soybean protein isolate solution and a carboxymethyl konjac glucomannan solution;
S1.2: adding hydrochloric acid to the soybean protein isolate solution to adjust pH to 4, then adding amygdalin at a solid-liquid ratio of 1 g:330 mL, and performing ultrasonic processing at 180 W for 30 min to obtain an amygdalin mixture; and
S1.3: adding the carboxymethyl konjac glucomannan solution to the amygdalin mixture, stirring at a rate of 800 r/min for 2 h, and performing polymerization to obtain an amygdalin-loaded polymer, where a volume ratio of the amygdalin mixture to the carboxymethyl konjac glucomannan solution was 3:1;

### S2: Preparation of Hydroxyproline-Modified Chitosan

S2.1: adding chitosan to a 1% acetic acid solution at a solid-liquid ratio of 1 g:90 mL, and fully stirring for dissolving to obtain a chitosan solution;
S2.2: adding hydroxyproline to deionized water at a solid-liquid ratio of 1 g:20 mL, and fully stirring for dissolving to obtain a hydroxyproline solution;
S2.3: adding 1-ethyl-3-(3-dimethylaminopropyl) carbodiimide hydrochloride and N-hydroxysuccinimide to the hydroxyproline solution, performing stirring reaction in an ice-water bath at 3° C. for 2 h to obtain an activated hydroxyproline solution, where mass ratios of the 1-ethyl-3-(3-dimethylaminopropyl) carbodiimide hydrochloride and the N-hydroxysuccinimide to the hydroxyproline were 1:3 and 1:5, respectively; and
S2.4: adding the activated hydroxyproline solution to the chitosan solution while stirring, then adding triethylamine, adjusting pH to 8, continuing to perform stirring reaction for 20 h, and then performing dialysis with the deionized water and freeze-drying to obtain the hydroxyproline-modified chitosan, where a volume ratio of the activated hydroxyproline solution to the chitosan solution was 1:9;

### S3: Mixed Preparation of the Temperature-Sensitive Degradable Gel

S3.1: adding the hydroxyproline-modified chitosan prepared in step S2.4 to a 1% acetic acid solution at a solid-liquid ratio of 1 g:60 mL, and fully stirring for dissolving to obtain a hydroxyproline-modified chitosan solution;
S3.2: adding the polygalacturonic acid and the sodium β-glycerophosphate to the deionized water, respectively, and stirring until completely dissolved to prepare 1 wt % of a polygalacturonic acid solution and 1 wt % of a sodium β-glycerophosphate solution;
S3.3: stirring equal volumes of the hydroxyproline-modified chitosan solution, the polygalacturonic acid solution, and the sodium β-glycerophosphate solution in an ice-water bath at 4° C. for 30 min to obtain a mixed sol solution; and
S3.4: adding the autologous platelet-rich plasma and the amygdalin-loaded polymer prepared in step S1.3 to the mixed sol solution, fully stirring and mixing, and then performing homogenization processing in a homogenizer for 30 min to obtain the temperature-sensitive degradable gel, where a volume ratio of the autologous platelet-rich plasma to the mixed sol solution was 1:10, and a mass ratio of the amygdalin-loaded polymer to the autologous platelet-rich plasma was 5:1; and preparation of the autologous platelet-rich plasma included the following steps: first, collecting peripheral blood of a patient for anticoagulation treatment, and then performing centrifugation to separate the supernatant, then adding thrombin and 10 wt % of a calcium chloride solution to a percentage content of the calcium chloride solution in the autologous platelet-rich plasma was 12%, and a content of the thrombin therein was 25 U/mL.

### Comparative Example 1

This Comparative Example 1 differed from Example 1 in that the amygdalin-loaded polymer in step S3.4 was removed.

### Comparative Example 2

This Comparative Example 2 differed from Example 1 in that the hydroxyproline-modified chitosan in step S3.1 was replaced with an equal amount of chitosan.

### Comparative Example 3

This Comparative Example 3 differed from Example 1 in that the amygdalin in step S1.2 was replaced with an equal amount of autologous platelet-rich plasma.

### Comparative Example 4

This Comparative Example 4 differed from Example 1 in that the autologous platelet-rich plasma in step S3.4 was replaced with an equal amount of amygdalin.

### Test Examples

Test 1: the temperature-sensitive degradable gels prepared in Examples 1-3 and Comparative Example 1 were heated to 37° C. to form gel bodies; the gel bodies were then cut into 10 mm×10 mm×10 mm cubes; and then, a universal material testing machine was used to test compressive strengths of the cubes, with results shown in Table 1.

TABLE 1
Comparison table of compressive strength test results

| | Compressive strength (MPa) |
|---|---|
| Example 1 | 0.92 |
| Example 2 | 0.94 |
| Example 3 | 0.95 |
| Comparative Example 1 | 0.46 |

It can be seen from Table 1 that the temperature-sensitive degradable gel prepared without adding the amygdalin-loaded polymer in Comparative Example 1 had a compressive strength of about 0.46 MPa, which was much less than that of Example 1. It can thus be concluded that soybean protein isolates and carboxymethyl konjac glucomannan were first prepared into solutions, respectively, amygdalin was added to the soybean protein isolate solution, and then a resulting mixture was mixed with the carboxymethyl konjac glucomannan solution for electrostatic self-assembly polymerization to form a carrier with a three-dimensional macromolecular structure, the amygdalin was loaded, and thus an amygdalin-loaded polymer was obtained. After the amygdalin-loaded polymer was added to the gel, the compressive strength of the gel can be effectively enhanced, thereby achieving the effect of improving the support force of the gel.

Test 2:2 mL of each of the temperature-sensitive degradable gels prepared in Examples 1-3 and Comparative Example 2 were placed in penicillin vials at room temperature for 30 min, and then were placed in a water bath at 37° C., timing was started, and the flow of the gels in the vials was observed. When the temperature-sensitive degradable gels in the vials did not flow with the tilt of the penicillin vials, the timing was stopped, and this duration was gelation time. Each sample was measured three times and a mean value was taken.

TABLE 2
Comparison table of gelation time test results:

| | | | | Comparative |
|---|---|---|---|---|
| | Example 1 | Example 2 | Example 3 | Example 2 |
| Gelation time (s) | 77 | 77 | 78 | 183 |

It can be seen from Table 2 that when chitosan was not modified by hydroxyproline in Comparative Example 2, the prepared temperature-sensitive degradable gel had the gelation time of about 183 s at 37° C., which was much greater than that of Example 1. It can thus be concluded that through operations that hydroxyproline and chitosan were first dissolved, respectively, hydroxyproline was then activated using 1-ethyl-3-(3-dimethylaminopropyl) carbodiimide hydrochloride and N-hydroxysuccinimide, and then the activated hydroxyproline solution was mixed with the chitosan solution for reaction, chitosan was modified; and after hydroxyproline-modified chitosan was added to the gel, the rapid gel-formation ability of the gel can be improved.

Test 3: seven-week-old adult female BALB/c mice were selected; clinical mechanical curettage injury was simulated in each mouse using a scraping method; each mouse was anesthetized with inhaled 5% isoflurane; the abdominal cavity of each mouse was accessed through the dorsal abdomen, thus exposing the uterine horn; a 24G needle was used to repeatedly rotate to scrape off the endometrium, while uterine congestion and thinning was observed under a stereomicroscope; the scraping operation was stopped when a roughness sensation was detected upon the needle contacting the uterine surface; the wound on the uterine surface caused by the needle was sutured using a sterile absorbable surgical suture; the abdominal cavity was closed layer by layer with postoperative absorbable sutures; and postoperatively, the mice were placed on a 37° C. heating pad and observed for 2 h. If no abnormalities were detected, the mice were returned to an animal room. After the mice recovered from anesthesia, water and mouse food were provided, and thus sample mice with intrauterine adhesion were obtained. Subsequently, the sample mice were divided into three groups: the first group was Example 1 Group, and 50 μL of the temperature-sensitive degradable gel prepared in Example 1 was injected in situ into the uterine cavity of each mouse; the second group was Comparative Example 3 Group, and 50 μL of the temperature-sensitive degradable gel prepared in Comparative Example 3 was injected in situ into the uterine cavity of each mouse; and the third group was Comparative Example 4 Group, and 50 μL of the temperature-sensitive degradable gel prepared in Comparative Example 4 was injected in situ into the uterine cavity of each mouse. After 14 days of the injection, Masson trichrome staining was performed on the three groups of mice, and the uterine fibrosis areas of the mice were analyzed, with results shown in Table 3.

TABLE 3
Comparison table of analysis results
for uterine fibrosis areas of mice

| | | Comparative | Comparative |
|---|---|---|---|
| | Example 1 | Example 3 | Example 4 |
| Uterine fibrosis | 26.8 | 49.3 | 75.6 |
| area (%) | | | |

It can be seen from Table 3 that when only autologous platelet-rich plasma was added in Comparative Example 3 and only amygdalin was added in Comparative Example 4, the uterine fibrosis areas of the mice with intrauterine adhesion were both greater than that of Example 1 after 14 days of injection with the prepared temperature-sensitive degradable gels. It can thus be concluded that the autologous platelet-rich plasma and amygdalin can synergistically treat the intrauterine adhesion, thus further improving the effect of treating the intrauterine adhesion.

The above examples are only intended to exemplarily illustrate the principle and effects of this disclosure but not intended to limit this disclosure. Any person of ordinary skill in the art can modify or change the above examples without departing from the spirit and scope of this disclosure. Therefore, all equivalent modifications or changes made by those of ordinary skill in the art without departing from the spirit and technical ideal disclosed by this disclosure should still fall within the claims of this disclosure.

What is claimed is:

1. A preparation method of a temperature-sensitive degradable gel for treating intrauterine adhesion, comprising the following steps:
S1: preparation of an amygdalin-loaded polymer
preparing a soybean protein isolate solution and a carboxymethyl konjac glucomannan solution from soybean protein isolates and carboxymethyl konjac glucomannan, respectively, adding amygdalin to the soybean protein isolate solution, and then mixing with the carboxymethyl konjac glucomannan solution for polymerization to obtain the amygdalin-loaded polymer;
S2: preparation of hydroxyproline-modified chitosan
dissolving and activating hydroxyproline, then adding to an acetic acid solution of chitosan, followed by adding triethylamine and performing stirring reaction to obtain the hydroxyproline-modified chitosan; and
S3: mixed preparation of the temperature-sensitive degradable gel
dissolving polygalacturonic acid, sodium β-glycerophosphate, and the hydroxyproline-modified chitosan, respectively, stirring and mixing same in an ice-water bath, then adding autologous platelet-rich plasma and the amygdalin-loaded polymer, and performing homogeneous mixing to obtain the temperature-sensitive degradable gel;
preparation of the autologous platelet-rich plasma comprising the following steps: first, collecting peripheral blood of a patient for anticoagulation treatment, and then performing centrifugation to separate the peripheral blood into an upper layer, a middle layer, and a lower layer; subsequently, aspirating the upper layer and the middle layer for secondary centrifugation; discarding supernatant, then adding thrombin and 8-10 wt % of a calcium chloride solution to a remaining portion to obtain the autologous platelet-rich plasma, wherein a volume percentage content of the calcium chloride solution in the autologous platelet-rich plasma is 8-12%, and a content of the thrombin therein is 15-25 U/mL.

2. The preparation method of a temperature-sensitive degradable gel for treating intrauterine adhesion according to claim 1, wherein S1 comprises the following steps:
S1.1: adding the soybean protein isolates and the carboxymethyl konjac glucomannan to distilled water respectively at a material-liquid ratio of 1 g:(90-100) mL, heating and stirring at 35-45° C. for dissolving while maintaining temperature, so as to obtain the soybean protein isolate solution and the carboxymethyl konjac glucomannan solution;
S1.2: adding hydrochloric acid to the soybean protein isolate solution to adjust pH to 3-4, then adding the amygdalin at a solid-liquid ratio of 1 g:(320-330) mL, and performing ultrasonic processing at 160-180 W for 20-30 min to obtain an amygdalin mixture; and
S1.3: adding the carboxymethyl konjac glucomannan solution to the amygdalin mixture, stirring at a rate of 700-800 r/min for 1-2 h, and performing polymerization to obtain the amygdalin-loaded polymer.

3. The preparation method of a temperature-sensitive degradable gel for treating intrauterine adhesion according to claim 2, wherein S2 comprises the following steps:
S2.1: adding the chitosan to a 1% acetic acid solution at a solid-liquid ratio of 1 g:(80-90) mL, and fully stirring for dissolving to obtain a chitosan solution;
S2.2: adding the hydroxyproline to deionized water at a solid-liquid ratio of 1 g:(10-20) mL, and fully stirring for dissolving to obtain a hydroxyproline solution;
S2.3: adding 1-ethyl-3-(3-dimethylaminopropyl) carbodiimide hydrochloride and N-hydroxysuccinimide to the hydroxyproline solution, performing stirring reaction in an ice-water bath at 1-3° C. for 1-2 h to obtain an activated hydroxyproline solution; and
S2.4: adding the activated hydroxyproline solution to the chitosan solution while stirring, then adding the triethylamine, adjusting pH to 7-8, continuing to perform stirring reaction for 16-20 h, and then performing dialysis with the deionized water and freeze-drying to obtain the hydroxyproline-modified chitosan.

4. The preparation method of a temperature-sensitive degradable gel for treating intrauterine adhesion according to claim 3, wherein S3 comprises the following steps:
S3.1: adding the hydroxyproline-modified chitosan prepared in step S2.4 to the 1% acetic acid solution at a solid-liquid ratio of 1 g:(50-60) mL, and fully stirring for dissolving to obtain a hydroxyproline-modified chitosan solution;
S3.2: adding the polygalacturonic acid and the sodium β-glycerophosphate to the deionized water, respectively, and stirring until completely dissolved to prepare 1 wt % of a polygalacturonic acid solution and 1 wt % of a sodium β-glycerophosphate solution;
S3.3: stirring equal volumes of the hydroxyproline-modified chitosan solution, the polygalacturonic acid solution, and the sodium β-glycerophosphate solution in an ice-water bath at 2-4° C. for 20-30 min to obtain a mixed sol solution; and
S3.4: adding the autologous platelet-rich plasma and the amygdalin-loaded polymer prepared in step S1.3 to the mixed sol solution, fully stirring and mixing, and then performing homogenization processing in a homogenizer for 20-30 min to obtain the temperature-sensitive degradable gel.

5. The preparation method of a temperature-sensitive degradable gel for treating intrauterine adhesion according to claim 2, wherein a volume ratio of the amygdalin mixture to the carboxymethyl konjac glucomannan solution is (2-3): 1.

6. The preparation method of a temperature-sensitive degradable gel for treating intrauterine adhesion according to claim 3, wherein mass ratios of the 1-ethyl-3-(3-dimethylaminopropyl) carbodiimide hydrochloride and the N-hydroxysuccinimide to the hydroxyproline are 1:(2-3) and 1:(3-5), respectively, and a volume ratio of the activated hydroxyproline solution to the chitosan solution is 1:(8-9).

7. The preparation method of a temperature-sensitive degradable gel for treating intrauterine adhesion according to claim 4, wherein a volume ratio of the autologous platelet-rich plasma to the mixed sol solution is 1:(8-10), and a mass ratio of the amygdalin-loaded polymer to the autologous platelet-rich plasma is (3-5): 1.

8. A temperature-sensitive degradable gel for treating intrauterine adhesion, wherein the temperature-sensitive degradable gel is prepared by the preparation method of a temperature-sensitive degradable gel for treating intrauterine adhesion according to claim 1.
